(19) United States
(12) Patent Application Publication
(10) Pub. No.: US 2025/0246284 A1
(43) Pub. Date: Jul. 31, 2025

(54) MALARIA INTERVENTION DRONE

(71) Applicant: Shubhan Bhattacharya, Cary, NC (US)

(72) Inventor: Shubhan Bhattacharya, Cary, NC (US)

(21) Appl. No.: 19/038,014

(22) Filed: Jan. 27, 2025

Related U.S. Application Data
(60) Provisional application No. 63/625,317, filed on Jan. 26, 2024.

(51) Int. Cl. G16H 20/13; B64U 20/87; B64U 101/55; G06T 7/37; G06V 10/58; G06V 20/17

(52) U.S. Cl. CPC G16H 20/13; B64U 20/87; G06T 7/37; G06V 10/58; G06V 20/17; B64U 2101/55; B64U 2201/10; G06T 2207/10036

(57) ABSTRACT

Various examples are provided related to malaria intervention. In one example, a system for malaria intervention includes a drone; an imaging system affixed to the drone; a treatment dispensing system affixed to the drone; and control circuitry configured to control dispensing of the treatment by the treatment dispensing system based at least in part upon analysis of the acquired multispectral imagery. The imaging system can acquire multispectral imagery and the treatment dispensing system can dispense a treatment. In another example, a method includes detecting one or more potential mosquito breeding site based upon analysis of multispectral imagery of an area; determining a flight plan comprising a sequence of targeted areas for application of a treatment based upon the one or more potential mosquito breeding site; and initiating autonomous operation of a drone along the flight plan to dispense the treatment.

### CROSS REFERENCE TO RELATED APPLICATIONS

This application claims priority to, and the benefit of, U.S. provisional application entitled “Malaria Intervention Drone” having Ser. No. 63/625,317, filed Jan. 26, 2024, which is hereby incorporated by reference in its entirety.

### BACKGROUND

Malaria is a life-threatening disease transmitted through bites of infected Anopheles mosquitoes and disproportionately affects marginalized populations. Despite global efforts to eradicate it, malaria remains a significant public health challenge, with 249 million cases in 2022 and the geographical expansion of malaria further threatens global health security. In 2023, the US recorded the first locally acquired malaria cases in decades. Effective control strategies include Larval Source Management (LSM) at breeding grounds to reduce Anopheles larvae, thus reducing adult population. However, identifying and treating Anopheles habitats by land surveys is difficult and expensive.

### SUMMARY

Aspects of the present disclosure are related to malaria intervention. In one aspect, among others, a system for malaria intervention comprises a drone; an imaging system affixed to the drone, the imaging system configured to acquire multispectral imagery; a treatment dispensing system affixed to the drone, the treatment dispensing system configured to dispense a treatment; and control circuitry configured to control dispensing of the treatment by the treatment dispensing system based at least in part upon analysis of the acquired multispectral imagery. In one or more aspects, the imaging system can be configured to acquire RGB and near-IR (NIR) imagery. The imaging system can be a dual-camera system comprising a RGB camera and a NoIR camera configured to capture synchronized images. The RBG camera and the NOIR camera can be mounted in a side-by-side arrangement. A red channel from the NOIR camera can provide the NIR imagery. The RGB and NIR imagery can be aligned utilizing a Fourier-based alignment. In various aspects, processing or computing circuitry can be configured to analyze the RGB and NIR imagery to detect one or more potential mosquito breeding site. The analysis can be based upon Normalized Difference Water Index (NDWI) following Otsu and Canny filtering of the RGB and NIR imagery. The treatment can be dispensed through a misting nozzle. The treatment can be a larvicide. In some aspects, the drone can be an autonomous drone configured to follow a flight plan. The flight plan can comprise a sequence of targeted areas for application of the treatment. The flight plan can be remotely adjusted mid-flight.

In another aspect, a method for malaria intervention, comprises detecting one or more potential mosquito breeding site based upon analysis of multispectral imagery of an area; determining a flight plan comprising a sequence of targeted areas for application of a treatment based upon the one or more potential mosquito breeding site; and initiating autonomous operation of a drone along the flight plan, the drone comprising a treatment dispensing system configured to dispense the treatment at the sequence of targeted areas. In one or more aspects, the multispectral imagery can comprise RGB and near-IR (NIR) imagery. The method can comprise obtaining the multispectral imagery with an imaging system affixed to the drone. The analysis of the multispectral imagery can be based upon Normalized Difference Water Index (NDWI) following Otsu and Canny filtering of the RGB and NIR imagery. In various aspects, the flight plan can be uploaded to the drone via a wireless connection. The method can comprise adjusting the flight plan mid-flight.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a malaria intervention drone (MALIntDrone) system, in accordance with various embodiments of the present disclosure.

FIGS. 2A and 2B illustrate examples of Normalized Difference Water Index (NDWI) count distributions before and after Otsu and Canny Filtering process, in accordance with various embodiments of the present disclosure.

FIG. 3 illustrates an example of a prototype autonomous drone, in accordance with various embodiments of the present disclosure.

FIG. 4 is a schematic diagram illustrating an example of components and connections of the autonomous drone, in accordance with various embodiments of the present disclosure.

FIG. 5 includes images showing a custom-built multispectral camera system, in accordance with various embodiments of the present disclosure.

FIG. 6 is a spectral transmission chart of a Roscolux #2007 filter, in accordance with various embodiments of the present disclosure.

FIGS. 7A and 7B are images illustrating before and after a Fourier-Based image alignment process based on the phase correlation property, in accordance with various embodiments of the present disclosure.

FIGS. 8A and 8B illustrate an example of anopheles larvae habitat detection for Kasungu District, in accordance with various embodiments of the present disclosure.

### DETAILED DESCRIPTION

Disclosed herein are various examples related to malaria intervention. Project MALIntDrone aims to develop an automated MALaria INTervention system to detect and treat larval habitats. Reference will now be made in detail to the description of the embodiments as illustrated in the drawings, wherein like reference numbers indicate like parts throughout the several views.

Malaria is a leading global public health crisis that disproportionately impacts marginalized populations: those least able to afford preventive measures and medical treatment. In 2022, the CDC reported an estimated 608,000 malaria deaths in 85 countries. Malaria is a leading cause of death and disease in many developing countries, where young children and pregnant women are the groups most affected due to their lowered immunity. It is both a cause and a consequence of poverty and, thus, a right-to-health issue.

The two primary interventions for Malaria vector control are insecticide-treated nets (ITNs), bed nets with insecticide to repel mosquitoes, and indoor residual spraying (IRS), coating of surfaces with insecticide. However, these methods face challenges due to insecticide resistance and altered mosquito behavior, including earlier biting times. Larval source management (LSM), a promising supplementary technique, involves managing mosquito breeding sites to reduce larvae numbers. In recent studies, LSM has been shown to reduce the number of new malaria cases by 49%-74% and malaria prevalence by 68%-89%. Additionally, LSM can protect disadvantaged populations who cannot afford core intervention strategies such as ITNs and IRS. A commonly used larvicide, Bacillus thuringiensis israelensis (BTI), shows no signs of resistance development in mosquitoes.

However, LSM is labor-intensive and costly, requiring regular ground treatment and detection of larval habitats. These are the primary factors hindering the widespread adoption of LSM in some African countries.

This work is focused on addressing these challenges and aims to develop an automated drone system for identification and treatment of potential Anopheles larval habitats, allowing large-scale LSM implementation. High-resolution satellite-based remote sensing techniques can be utilized to provide insights on potential Anopheles habitats and then an autonomous drone can be deployed to spray BTI on these locations. There is particular interest in detecting aquatic vegetation, known to be a source of anopheles mosquito breeding sites and contain a high prevalence of anopheles larvae.

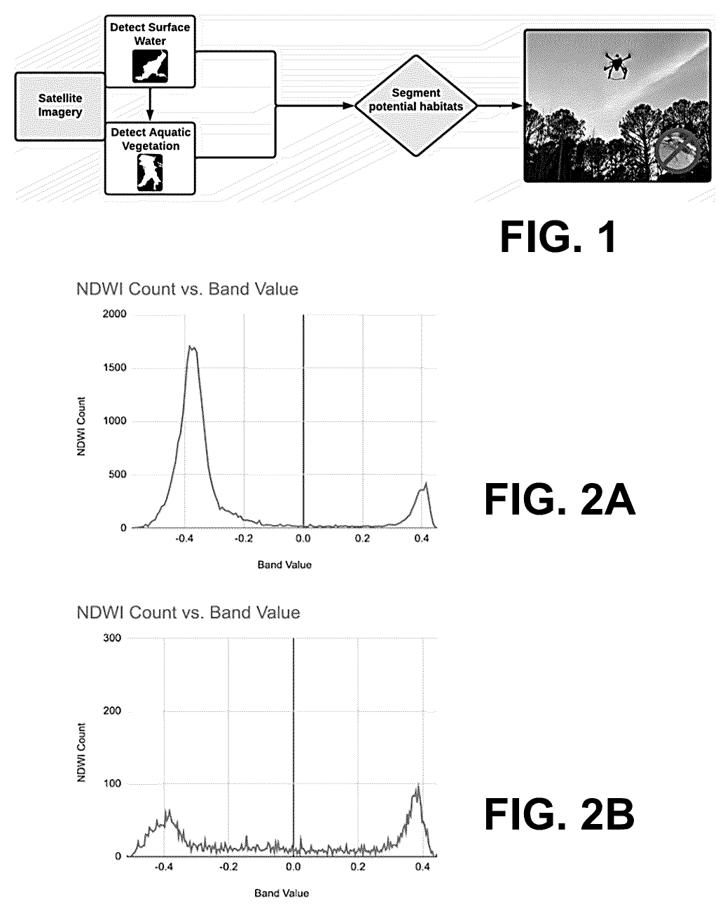

FIG. 1 shows a comprehensive overview of the MALIntDrone: from the detection of Anopheles larvae habitats using remote sensing to the deployment of a custom-made autonomous drone. Advanced remote sensing techniques can be utilized to identify Anopheles habitats: Normalized Difference Water Index (NDWI) with Otsu thresholding and Canny edge filtering for water bodies and Normalized Difference Aquatic Vegetation Index (NDAVI) and Water Adjusted Vegetation Index (WAVI) for aquatic vegetation. MALIntDrone achieved 85% accuracy for detecting Anopheles larvae habitats and 86% for aquatic vegetation sites using ground surveyed data from the Kasungu District, Malawi. To address potential limitations of satellite imagery, an inexpensive multispectral camera was developed using a dual-camera Raspberry Pi system to provide RGB+Near-IR imagery which can then be aligned using a Fourier-Based image alignment. This camera can be attached to the drone to be used to create orthomosaics of an area. To treat areas effectively, a developed autonomous drone with a precision sprayer system can target identified habitats. MALIntDrone's multidisciplinary approach has the potential to impact the health of millions worldwide as it is easy to use and efficient and can aid the WHO's goal of Malaria eradication by 2030.

### Detecting Breeding Sites

The focus was on detecting areas of aquatic vegetation as potential breeding sites for Anopheles Mosquitos. It was decided to first detect surface water and then further segment aquatic vegetation. The RGB bands and Near-IR (NIR) bands of satellite imagery were only utilized in this work as they provided the highest resolution imagery.

Surface Water. In this section, the methods employed for surface water detection are detailed. This work incorporates three key techniques: the Normalized Difference Water Index (NDWI), Otsu thresholding, and Canny filtering.

(1) NDWI: The NDWI is used to maximize the variance between water and non-water features from an image. The index uses the reflectance in the green and NIR bands.

$$N D W I = \frac{Green - N I R}{Green + N I R} \qquad ( 1 )$$

The resulting value enhances the presence of water bodies from other land cover types in the satellite imagery.

(2) Otsu thresholding: While the NDWI enhances the presence of water bodies, it cannot be applied with a single global threshold due to varying spectral properties of land in different geographic areas. To address this shortcoming, the Otsu method, a histogram-based thresholding technique, was utilized. This method is used to maximize the variance between the two classes (water and non-water) by finding an optimal local threshold value.

Otsu's algorithm assumes a bimodal distribution and calculates the threshold that minimizes the intra-class variance. This step significantly improves the segmentation accuracy of surface water by incorporating local thresholds.

(3) Canny filtering: Yet Otsu's algorithm presents its own shortcomings. Since it assumes a bimodal distribution it can perform inaccurately when presented with a non-bimodal distribution.

$${K}_{x} = \{ \begin{matrix}- 1 & 0 & 1 \\ - 2 & 0 & 2 \\ - 1 & 0 & 1\end{matrix} \} , {K}_{y} = \{ \begin{matrix}1 & 2 & 1 \\ 0 & 0 & 0 \\ - 1 & - 2 & - 1\end{matrix} \} \qquad ( 2 )$$

Thus, further refinement of the image can be performed using Canny edge filtering. The Canny edge detection algorithm is applied to attempt to detect the edges of water bodies.

This method involves: applying a Gaussian filter to smooth the image, calculating the gradient intensity in the X and Y direction using the Sobel operator (Kₓ, K_{y}) eq. (2), and using non-maximum suppression to thin the edges. The result after the filter can be seen in FIGS. 2A and 2B. FIG. 2A is a histogram representing NDWI over an area and FIG. 2B is a histogram representing NDWI following Otsu and Canny filtering over an area.

Detecting aquatic vegetation. In this section, the methodology employed for further detecting aquatic vegetation from surface water is discussed. The approach utilizes the Water Adjusted Vegetation Index (WAVI) and the Normalized Difference Aquatic Vegetation Index (NDAVI), which have been shown to effectively separate aquatic vegetation.

The formulas for these indices are as follows:

$$N D A V I = \frac{N I R - Blue}{N I R + Blue} \qquad ( 3 )$$

$$W A V I = \frac{( N I R - Blue ) ( 1 + L )}{N I R + Blue + L} \qquad ( 4 )$$

The NDAVI and WAVI index found that the NIR band and the blue band can be used in order to spectrally separate terrestrial vegetation and aquatic vegetation. NIR is also absorbed by water, so the NDAVI and WAVI indices can spectrally separate aquatic vegetation and water.

The WAVI index includes a correction factor, L, that is formulated to adjust for the influence of a water background. Multiplying by (1+L) and dividing by L increases the sensitivity of the index to vegetation presence. It helps to amplify the vegetation signal relative to the water signal. For our study, a suggested value of L=0.5 was utilized.

Once surface water was segmented, the WAVI and NDAVI indices were applied. Higher values from these indices were highlighted with a higher intensity red masking, making it easier to distinguish aquatic vegetation.

### Drone Building

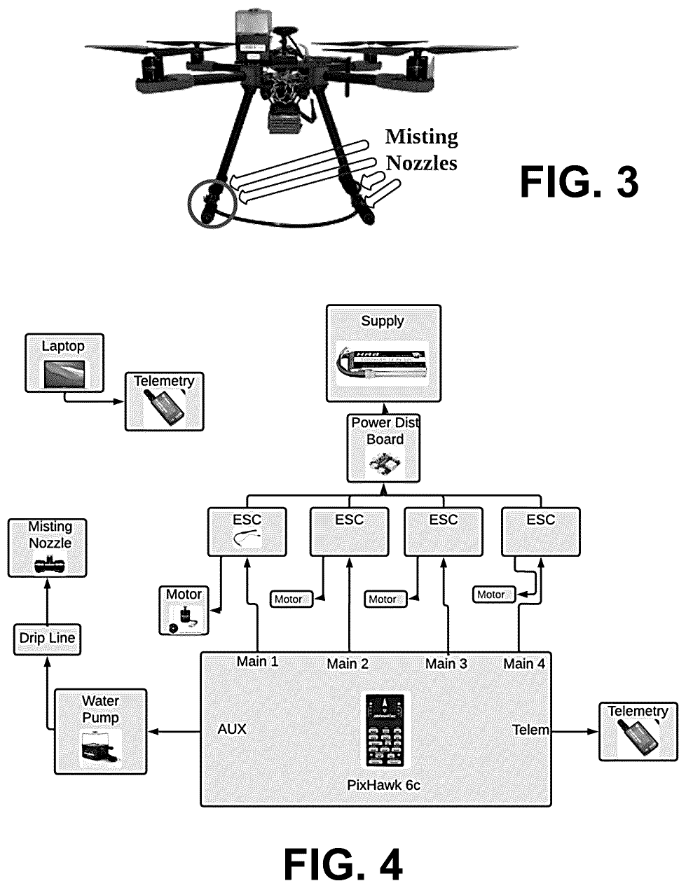

Autonomous Drone Design. FIG. 3 shows a prototype autonomous drone designed and developed for the MALIntDrone project. The drone was built using a 500 mm frame equipped with a Pixhawk 6c flight controller. This controller was chosen for its advanced autonomous flight capabilities and compatibility with various sensors. The drone is powered by a 14.8V 4S Lipo Battery 5000 mAh (sufficient for an estimated 25-35 minute mission). The drone was equipped with a custom sprayer system with a water pump and misting nozzles to dispense larvicide efficiently. The drone configuration includes a custom built sprayer system including a water pump and four misting nozzles (at each leg of the drone) to ensure uniform coverage of targeted areas as shown in FIG. 3. FIG. 4 is a schematic diagram representing components and connections of the autonomous drone. ESC is an electronic speed control.

Flight Control. A flight plan for the drone can be designed using Ground-Control, an open-source ground control station software. This software allows for waypoint planning and mission management. The flight plan is then uploaded using Telemetry Radio, which allows for real time monitoring of the drone. This ensures the drone can be controlled remotely and adjustments can be made mid-flight if necessary.

### Design of Multispectral Camera

Cloud cover and forest cover can significantly impede useful satellite imagery, which is important for identifying potential breeding sites. To overcome this challenge, an inexpensive multispectral camera that can be deployed on the drone was developed to provide RGB and NIR imagery even when satellite data is unavailable. Images taken from the drone when using the multispectral camera can be at sufficient height (e.g., at least 10 meters) to create an orthomosaic of an area, thus it will not cause any modification or disturbance to the mosquito breeding ground.

Dual Camera system. The multispectral camera system utilizes a Raspberry Pi 4 and consists of two cameras to capture both RGB and NIR channels. These can then be aligned using Fourier based process. The components include:
A Raspberry Pi Camera Module V2-8 Megapixel, 1080p
Raspberry Pi NoIR Camera Module V2-8MP 1080P30

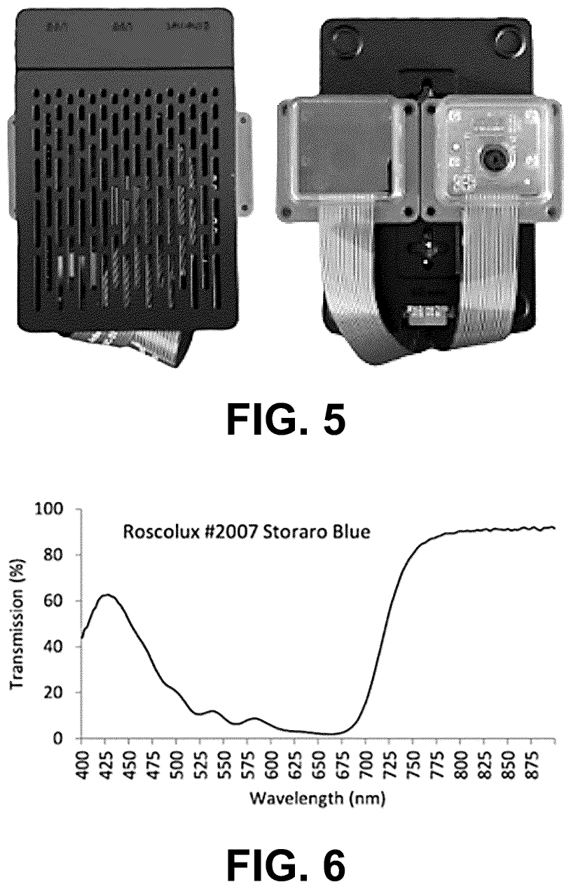

FIG. 5 includes images showing the custom-built multispectral camera system with the front of multispectral camera on the left and the back of multispectral camera on the right. The NoIR camera has no infrared filter which can be combined with a blue filter (Roscolux #2007) to get NIR reflectance. FIG. 6 is a spectral transmission chart of the Roscolux #2007 filter used for the custom-built multispectral camera system, which shows high transmission in the NIR range. As a result, the NIR data is stored as the red channel in the captured image.

Fourier-Based Image Alignment. To align the images from the two cameras, Fourier-based alignment using Wolfram Mathematica can be employed. This process ensures that the images are correctly aligned by using the phase correlation property of the Fourier transform as defined below.

$$P C = \{ \frac{( u , v ) \cdot ( u , v )}{\vert ( u , v ) \cdot ( u , v ) \vert} \} \qquad ( 5 )$$

where:
PC denotes the Phase Correlation;
[symbol] denotes the inverse Fourier Transform;
[symbol] (u, v) is the Fourier Transform (FT) of the first image;
[symbol] (u, v) is the Fourier Transform (FT) of the second image; and
[symbol] (u, v) represents the complex conjugate of [symbol] (u, v).

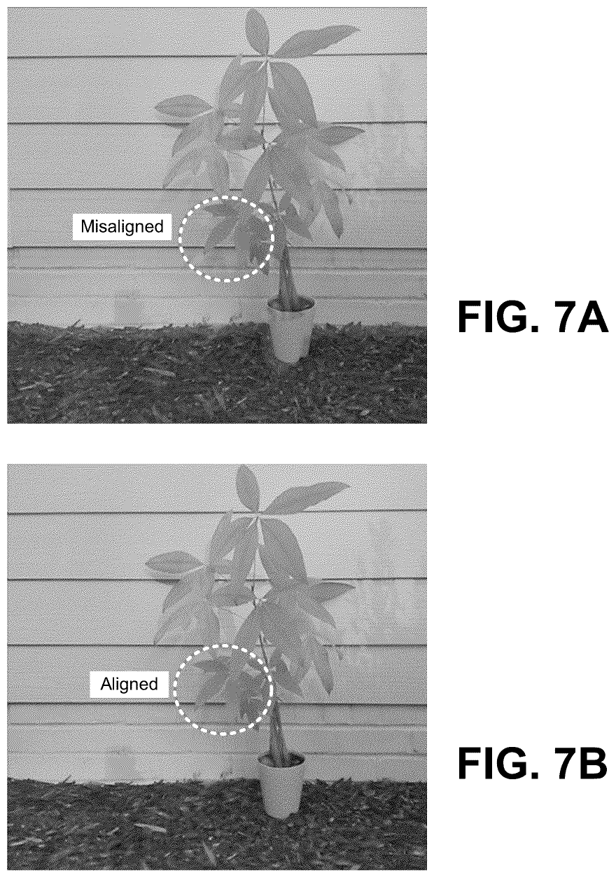

The peak from results of eq. (5) can be then used to align the two images, as it corresponds to where the overlap between the two images is maximal. Once the images are aligned the red channel from the NOIR camera is used as the NIR band. The RGB bands from the RGB camera and the NIR band can be used to perform various index calculations, as given in eq. (3) and eq. (4). FIG. 7A shows an example of an initial overlay of misaligned images from the RGB and NoIR cameras and FIG. 7B shows a final aligned overlay of the images from the RGB and NoIR cameras following the Fourier transform process.

### Results

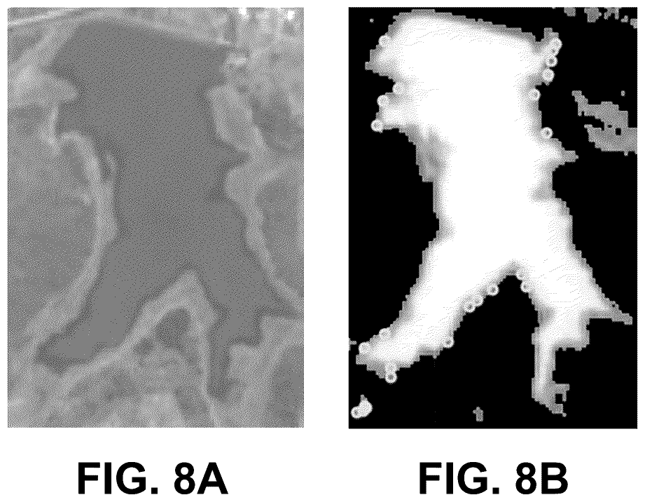

This section presents the results of the approach for detecting potential Anopheles larvae habitats. The data used to evaluate the approach was provided through an open access ground entomological survey conducted from 2018 to 2020 in the Kasungu district, Malawi. FIG. 8A is an RGB satellite image of the site in Kasungu District, Malawi. A total of 143 aquatic vegetation points were used, out of which 52 were classified as Anopheles larvae habitats. These areas were evaluated using 10 meter Sentinel 2 satellite imagery from the corresponding time period and compared with the ground sampled results. FIG. 8B illustrates the results of detection. Detected potential habitats are highlighted by shading and circles represent ground sampled sites from the entomological survey.

The approach employed successfully identified Anopheles mosquito habitats and aquatic vegetation with high accuracy, achieving an 85% accuracy rate for Anopheles larvae habitat detection and an 86% accuracy rate for aquatic vegetation detection. Results of the anopheles larvae habitat detection is shown in the following table.

Anopheles Larvae Habitat Detection

| | |
|---|---|
| Total predicted correctly | 44 |
| Total habitats | 52 |
| Percentage | 85% |

Results of the aquatic vegetation detection are shown in the following table.

Aquatic vegetation (AV) detection

| | |
|---|---|
| Predicted correctly | 123 |
| Total AV points | 143 |
| Percentage | 86% |

These results are expected to improve further with higher resolution imagery, such as SkySat 50 cm subscription data, which could potentially lead to accuracy above 90%.

The multispectral camera developed was able to provide calibrated RGB and Near-IR imagery at a significantly lower cost ($130) compared to existing products (over $1500). Allowing for imaging even when satellite imagery is impeded by cloud cover.

The developed drone system presents a prototype for a relatively inexpensive drone for accurately spraying larvicide on designated waypoints. Environmental factors, such as severe weather conditions, can impact the drones operational efficiency. Heavy rainfall and strong winds can impede the drone's ability to fly safely and also capture accurate multispectral imagery. Weather radar APIs, can be incorporated to enable the drone to return safely during adverse weather, and utilize weather patterns for mission planning to optimize efficiency. Sealing the battery in a fire-safe casing, designated takeoff and landing sites, and the encasing of electronics can also be included.

The MALIntDrone project presents a prototype automated system for large-scale LSM application. By adopting a multidisciplinary approach that combines remote sensing, optics and electrical engineering, the project has created a novel system that can significantly impact the fight against Malaria. This method presents a cost effective and strategic method for large-scale LSM application. It can be used in local communities all across an area of high transmission to reduce Malaria prevalence.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A system for malaria intervention, comprising:
a drone;
an imaging system affixed to the drone, the imaging system configured to acquire multispectral imagery;
a treatment dispensing system affixed to the drone, the treatment dispensing system configured to dispense a treatment; and
control circuitry configured to control dispensing of the treatment by the treatment dispensing system based at least in part upon analysis of the acquired multispectral imagery.

2. The system of claim 1, wherein the imaging system is configured to acquire RGB and near-IR (NIR) imagery.

3. The system of claim 2, wherein the imaging system is a dual-camera system comprising a RGB camera and a NoIR camera configured to capture synchronized images.

4. The system of claim 3, wherein the RBG camera and the NOIR camera are mounted in a side-by-side arrangement.

5. The system of claim 3, wherein a red channel from the NOIR camera provides the NIR imagery.

6. The system of claim 2, wherein the RGB and NIR imagery are aligned utilizing a Fourier-based alignment.

7. The system of claim 2, wherein the RGB and NIR imagery are analyzed to detect one or more potential mosquito breeding site.

8. The system of claim 7, wherein the analysis is based upon Normalized Difference Water Index (NDWI) following Otsu and Canny filtering of the RGB and NIR imagery.

9. The system of claim 1, wherein the treatment is dispensed through a misting nozzle.

10. The system of claim 9, wherein the treatment is a larvicide.

11. The system of claim 1, wherein the drone is an autonomous drone configured to follow a flight plan.

12. The system of claim 11, wherein the flight plan comprises a sequence of targeted areas for application of the treatment.

13. The system of claim 11, wherein the flight plan is remotely adjusted mid-flight.

14. A method for malaria intervention, comprising:
detecting one or more potential mosquito breeding site based upon analysis of multispectral imagery of an area;
determining a flight plan comprising a sequence of targeted areas for application of a treatment based upon the one or more potential mosquito breeding site; and
initiating autonomous operation of a drone along the flight plan, the drone comprising a treatment dispensing system configured to dispense the treatment at the sequence of targeted areas.

15. The method of claim 14, wherein the multispectral imagery comprises RGB and near-IR (NIR) imagery.

16. The method of claim 15, comprising obtaining the multispectral imagery with an imaging system affixed to the drone.

17. The method of claim 16, wherein the analysis of the multispectral imagery is based upon Normalized Difference Water Index (NDWI) following Otsu and Canny filtering of the RGB and NIR imagery.

18. The method of claim 14, wherein the flight plan is uploaded to the drone via a wireless connection.

19. The method of claim 18, comprising adjusting the flight plan mid-flight.
